(19) United States
(12) Patent Application Publication
(10) Pub. No.: US 2006/0036230 A1
(43) Pub. Date: Feb. 16, 2006

(54) Shaped frontal patch

(76) Inventors: Michael W. Mills, Cottage Grove, MN (US); Mark A. Peltier, Forest Lake, MN (US)

Correspondence Address: 3M INNOVATIVE PROPERTIES COMPANY, PO BOX 33427, ST. PAUL, MN, 55133-3427, US

(21) Appl. No.: 10/918,038

(22) Filed: Aug. 13, 2004

(51) Int. Cl. A61F 13/15

(52) U.S. Cl. 604/391

(57) ABSTRACT

A disposable absorbent article comprising a chassis having a longitudinal centerline and an outwardly-facing surface and an inwardly-facing surface, the chassis adapted to extend from a back waist area of a wearer to a front waist area of a wearer with the inwardly-facing surface overlying the crotch area of the wearer, the chassis including a first front waist region, a second back waist region and a crotch region; and a fastening system comprising at least one first fastening element and a landing member engagable with the at least one first fastening element on opposing waist regions wherein the landing member has a top edge and a bottom edge wherein at least the top edge is nonlinear and provided with a multitude of upstanding features.

### BACKGROUND AND SUMMARY

1. Field of the Invention

The present invention relates to disposable absorbent articles, such as disposable diapers. More particularly, the present invention relates to a disposable absorbent article that includes an attachment panel for use with tape or mechanical type fastener tabs.

### BACKGROUND OF THE INVENTION

Infants and other incontinent individuals wear absorbent articles such as diapers and incontinent briefs to receive and contain discharged urine and other body exudates. Such absorbent articles function both to contain the discharged materials and to isolate those materials from the body of the wearer and from the wearer's garments and bed clothing. Disposable absorbent articles having many different basic designs are known in the art. Disposable absorbent articles are often supplied in a form that requires assembly of the article relative to the body of the wearer. Such articles generally include fastener tabs for securing the article in position about the waist of the wearer. These fastening tabs are generally adhesive tapes or mechanical fasteners (typically hook-type mechanical fasteners). The fastener is typically used with a mating attachment surface such as a tape landing zone or a loop material, that is attached to the front portion of the absorbent article by hot melt or pressure-sensitive adhesives. In order to attach these attachment surfaces to the absorbent article they are typically provided with a continuous backing such as a film backing. These backings can be somewhat rigid and have sharp edges that can create red marks on the wearer.

Also, proper application and positioning of a disposable diaper is important for the comfort of the wearer, and also for the proper functioning of the diaper. Unless one is especially careful in the course of applying a disposable diaper, it is very easy to apply the diaper to a wearer in a manner that results in the diaper not properly fitting to the wearer's body. For example, if the diaper is applied and fastened so that it is non-symmetrical, one of the leg openings that is formed by the longitudinal edges of the diaper, when the diaper is applied and is worn, is larger than the other leg opening. As a result, it is possible for unintended leakage to take place between the diaper leg opening and the wearer's leg on the side of the diaper having the larger leg opening. Additionally, the smaller leg opening might be too tight about the wearer's leg, causing discomfort. Thus, it is desirable to provide a means whereby the person applying a disposable diaper can do so simply and quickly, and in such a manner that the diaper is symmetrically and comfortably positioned on the wearer's body.

The matter of facilitating the proper application and fastening of a disposable diaper has been addressed by others. For example, in U.S. Pat. No. 4,662,875, entitled “Absorbent Article,” which issued on May 5, 1987, to Hirotsu et al., there is disclosed a positioning arrangement to aid in properly fitting a disposable diaper to a wearer. The disclosed positioning arrangement includes indicia that are provided on the outer face of the diaper backsheet in the areas to which the tape fastener tabs are secured during application of the diaper. The indicia illustrated in that patent include a plurality of parallel, spaced, longitudinally extending lines and a plurality of dots, each placed on and extending over a portion of the outer surface areas at which the fastener tabs are to be secured. However, this does not address the issue of red marks and wearer discomfort.

Another patent that discloses a tape fastener tab positioning arrangement is U.S. Pat. No. 5,897,546, entitled “Disposable Diaper Having a Fastening System,” which issued on Apr. 27, 1999, to Kido et al. The improvement disclosed by Kido et al. resides in placing positioning indicia between the diaper backsheet and an overlying fastener tab-landing zone. The indicia are provided by longitudinally extending strips of colored adhesive that serve to secure the landing zone strip in position on the backsheet. The color shows through the landing strip to provide placement guidance to one applying the diaper. Kido et al. point out that their approach does not require surface printing of the backsheet, and it also precludes contact by the wearer of the printed positioning indicia.

It is desirable to provide a fastening system that not only enables symmetrical application and placement of the fastener tabs but also provides a secure and comfortable attachment.

It is also desirable to provide an improved disposable absorbent article that includes a fastening system, wherein the attachment surface to which the fastener tabs or side panels are secured has features that facilitate positioning of the side panels or fastener tabs.

It would also be desirable to provide an absorbent article with a fastening system that is comfortable for the user and is also easy to attach to the absorbent article in a continuous manufacturing process.

### SUMMARY OF THE INVENTION

The invention relates to a disposable absorbent article having a landing member for a fastening element. The landing member has low compression resistance and can provide reference guides for fit.

The disposable article comprises a chassis having a longitudinal centerline and an outwardly-facing surface and an inwardly-facing surface. The chassis is adapted to extend from a back waist area of a wearer to a front waist area of a wearer with the inwardly-facing surface overlying the crotch area of the wearer. The chassis includes a first front waist region, a second back waist region and a crotch region and a fastening system. The fastening system comprises at least one first fastening element and a landing member engagable with the at least one first fastening element on opposing waist regions. The landing member has a top edge and a bottom edge wherein at least the top edge is nonlinear and provided with a multitude of upstanding features.

### BRIEF DESCRIPTION OF THE DRAWINGS

FIG. 1 is a perspective view of an absorbent article using the invention landing member.

FIG. 2 is a front view of a landing member in accordance with the invention.

FIG. 2a is an exploded view of the features on a top edge of the FIG. 2 landing member.

FIG. 3 is a second embodiment landing member in accordance with the present invention.

FIG. 3a is an exploded view of the features of the FIG. 3 embodiment.

FIG. 4 is a third alternative embodiment of the present invention landing member with features.

FIG. 4a is an exploded view of the features of the FIG. 4 embodiment.

FIG. 5 is a fourth embodiment of a landing member in accordance with the present invention.

FIG. 6 is a fifth embodiment of a landing member in accordance with the present invention.

FIGS. 7-12 are alternative embodiments of landing members in accordance with the present invention.

### DETAILED DESCRIPTION OF THE INVENTION

As used herein, the term “absorbent article” refers to devices which absorb and contain body exudates, and more specifically, refers to devices which are placed against or in proximity to the body of the wearer to absorb and contain the various exudates discharged from the body. The term “disposable” is used herein to describe absorbent articles which generally are not intended to be laundered or otherwise restored or reused as an absorbent article (i.e., they are intended to be discarded after a single use and, preferably, to be recycled, composted or otherwise disposed of in an environmentally compatible manner).

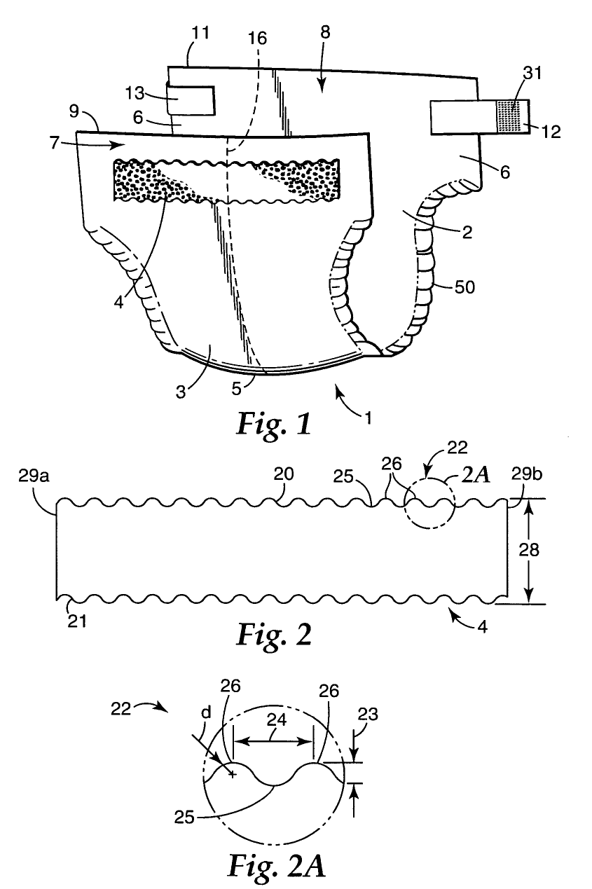

A preferred embodiment of an absorbent article of the present invention is the unitary disposable absorbent article, diaper 1, shown in FIG. 1. As used herein, the term “diaper” refers to an absorbent article generally worn by infants and incontinent persons about the lower torso. The present invention is also applicable to other absorbent articles such as incontinence briefs, incontinence undergarments, absorbent inserts, diaper holders and liners, feminine hygiene garments, bandages and the like.

FIG. 1 is a perspective view of the diaper 1 of the present invention. As shown in FIG. 1, the diaper 1 preferably comprises a liquid pervious topsheet 2; a liquid impervious backsheet 3; an absorbent core (not shown), which is preferably positioned between at least a portion of the topsheet 2 and the backsheet 3; side panels 6; and fastening tabs generally designated 12 and 13. Diaper 1 is shown in FIG. 1 to have a first waist region 8, a second front waist region 7 opposed to the first back waist region 8 and a crotch region 5 located between the first waist region and the second waist region. The periphery of the diaper 1 is defined by the outer edges of the diaper 1 in which the longitudinal side edges 50 run generally parallel to the longitudinal centerline 16 of the diaper 1 and the end edges 9, 11 run between the longitudinal side edges generally perpendicular to a longitudinal centerline of the diaper 1.

The backsheet 3 is generally that portion of the diaper 1 positioned adjacent the garment-facing surface of the absorbent core. Backsheet 3 prevents the exudates absorbed and contained therein from soiling articles that may contact the diaper 1, such as bedsheets and undergarments. In preferred embodiments, the backsheet 3 is impervious to liquids (e.g., urine) and comprises a thin plastic film such as a thermoplastic film having a thickness of about 0.012 mm (0.5 mil) to about 0.051 mm (2.0 mils).

The topsheet 2 is preferably positioned adjacent the body-facing surface of the absorbent core and may be joined thereto and/or to the backsheet 3 by any attachment means known in the art. The topsheet 2 is preferably compliant, soft feeling, and non-irritating to the wearer's skin. Further, at least a portion of the topsheet 2 is liquid pervious, permitting liquid to readily penetrate through its thickness. A suitable topsheet 2 may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. The nonwoven webs are formed into coherent webs by processes such as spunbonding, carding, wet-laying, meltblowing, hydroentangling, or other processes known in the art. Preferably, the topsheet 2 is made of a hydrophobic material or is treated to be hydrophobic in order to isolate the wearer's skin from liquids contained in the absorbent core.

The diaper 1 includes fastening tabs 12 and 13. The fastening tabs 12 and 13 preferably maintains the side panels 6 of the first waist region 8 and the second waist region 7 in at least partially overlapped condition to provide lateral tension about the circumference of the diaper 1 when it is worn, to hold the diaper 1 on the wearer.

The fastening tabs 12 and 13 comprises one or more fastening elements 31 that can be provided on a fastening tab, or could be provided on an ear or a portion of the side edge, that engage a landing member 4. These fastening elements 31 can be hook and/or loop fastening member components, adhesive member components, or any other known fastening means. The fastening element generally can be provided as adhesive tape tabs, or hook tabs in preferred embodiments. The landing member 4 can be a mating material to the fastening element such as a loop for use with hook fastening elements or a film-based substrate for an adhesive fastening element. Generally, the fastening element is that portion of the fastening tab or ear or the like, engagable with the landing member.

When diaper 1 is in its assembled form, longitudinally extending side edges 50 define leg openings that encircle the wearer's thighs to prevent leakage therearound. Elastic gasketing cuffs can be used to provide a further sealing effect around the wearer's thighs to prevent leakage. The side edges are preferably in contact with the wearer's thighs, and the contact pressure is in part determined by the orientation of diaper 1 on the body of the wearer and the respective orientation of the fastening elements such as on two fastener tabs 12 and 13 relative to the diaper longitudinal centerline 16. Thus, if the two fastener tabs 12 and 13 are each placed at significantly different distances from the longitudinal centerline 16 of diaper 1, the diaper will not be symmetrically positioned on the wearer's body, and therefore one of the leg openings will be larger than the other. By providing and utilizing the feature in accordance with the present invention, however, the problems noted above as well as others resulting from an ill-fitting diaper can be minimized.

One embodiment of a diaper in accordance with the present invention preferably includes a landing member 4 that has top and bottom edges 20 and 21 which has features 22, having peaks 26 and valleys 25, to facilitate proper positioning of fastening tabs 12 and 13. The fastener tabs optionally can have matching features and serve as a visual aid in positioning the fastener tabs symmetrically on the front waist area of the diaper, relative to the diaper longitudinal centerline.

The landing member 4 can be in the form of a pressure-sensitive adhesive tape, for attachment to the front portion of the diaper, or can be attached by other means such as use of hot melt adhesive, sonic welding or heat bonding. Preferably, the fastening landing member 4 has a continuous backing formed of a film or a consolidated fibrous web and a tensile strength of at least 0.5 kg/2.54 cm, generally at least 1.0 kg/2.54 cm. This allows the material to be handled in a continuous manufacturing process and can be easily applied either as a tape or otherwise attached to a substrate. Mechanical strength is desirable from a manufacturing standpoint. However, the backing thickness and strength provides that the landing member 4 be generally rigid which can cause irritation to the wearer in the waist region where it is applied. In a further aspect of the present invention the features 22 also create a soft edge such that when placed under compression it is easily deformed, eliminating or reducing the possibility of irritation or red marking of the wearer in the stomach region. The features are generally spaced at a distance 24 of at least 2 mm, preferably at least 5 mm to 100 mm where the height of the features 23 is generally at least 2 mm, preferably at least 3 mm where the ratio of the height of the feature to the width between peaks 26 of the landing member is from 0.02 to 10.0.0, or 0.03 to 5.0.

Referring to FIG. 2, there is shown one embodiment of a landing member 4 in accordance with the present invention. The top edge 20 includes features 22 in the form of regular, undulating structures or mounds. The features are generally symmetrical on either side of the centerline 16 so that an equal or substantially equal number of features are on each side of the centerline 16. The features have a height 23 measured from a peak 26 to a valley 25. The features are sometimes characterized by having radii of curvature, greater than 2 mm, generally greater than 3 mm. All of the features 22 on edge 20 could have this radius of curvature in some embodiments. The present invention can also be characterized by the lower edge 21 of the landing member 4 having features which can be the same or different than the features on the top edge 20. The greatest width 28 of the landing member is generally greater than or equal to the greatest width of the of the fastening tab element 31. The landing member generally has a backing having a Gurley Stiffness of from 5 to 500 or 10 to 500 and a compression stiffness of generally less 700 grams. The backing could be a thermoplastic film, a consolidated nonwoven or the like.

The features generally will have a height 23 measured from a peak to a valley which is a fraction of the width 28 of the landing member 4. Generally, this height 23 will be from 5 to 50 percent of the maximum landing member width 28 or from 10 to 40 percent of the maximum landing member width. The overall height 23 generally will be greater than at least 2 mm, preferably at least 3 mm. Features below this height will not be noticeable to a user to the extent that they are useable for symmetrical application of fastening elements as their function as softening elements for the outer edge of the landing member is significantly reduced. Generally, the features should have a height of at least 2 mm to allow for reduction of the compression stiffness to soften the edge of the landing member and to avoid the occurrence of red marking and irritation for the user. The compression stiffness of the landing member should be reduced (compared to a landing member without features), over at least a portion of the upper edge 26, by at least 15 percent, or 30 percent or more such as 50 percent. The reduction in compression stiffness is preferable at least in the central portion of the landing member 4.

The features generally will be substantially continuous across the upper edge of the landing member, however the features could be provided only in regions such as, for example, only in the central region where the amount of compression is the greatest and the need for reduced compression stiffness is most pronounced. If the features 22 are used solely for their softening effect, the regularity of the features is not as important and the features can be irregular or be asymmetrically located on either side of the centerline 16. Again, preferably, the features will have a height of at least 2 mm at least in the areas where reduced compression stiffness is needed when the landing zone is in use. The features can be of different heights and locations and can be either regularly or irregularly placed along one or both edges 20 or 21. The features can also be placed on one or both of the edges 29a and 29b. When used as a softening feature, generally the features will provide a compression resistance of less than 500 grams, or less than 250 grams.

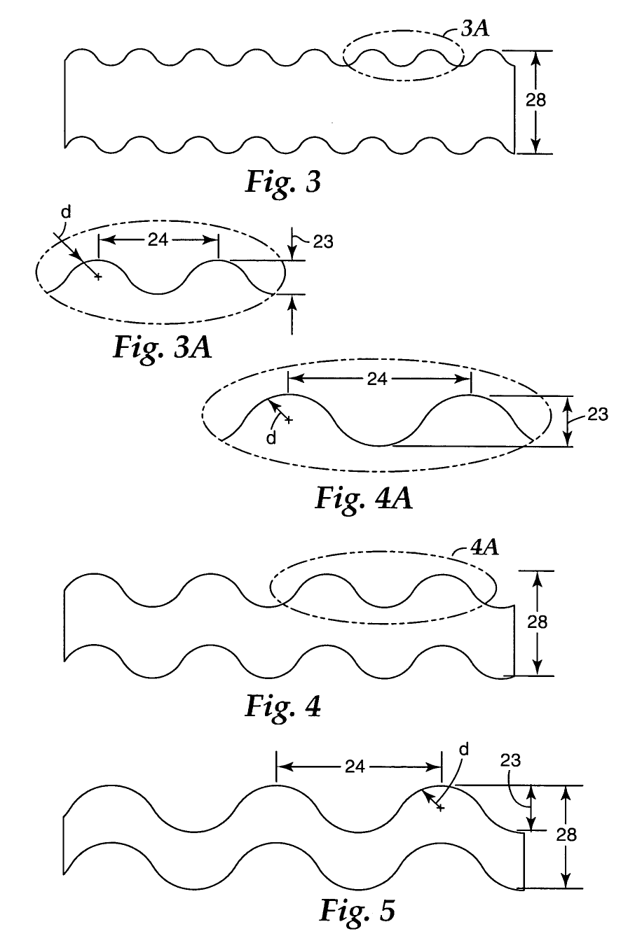

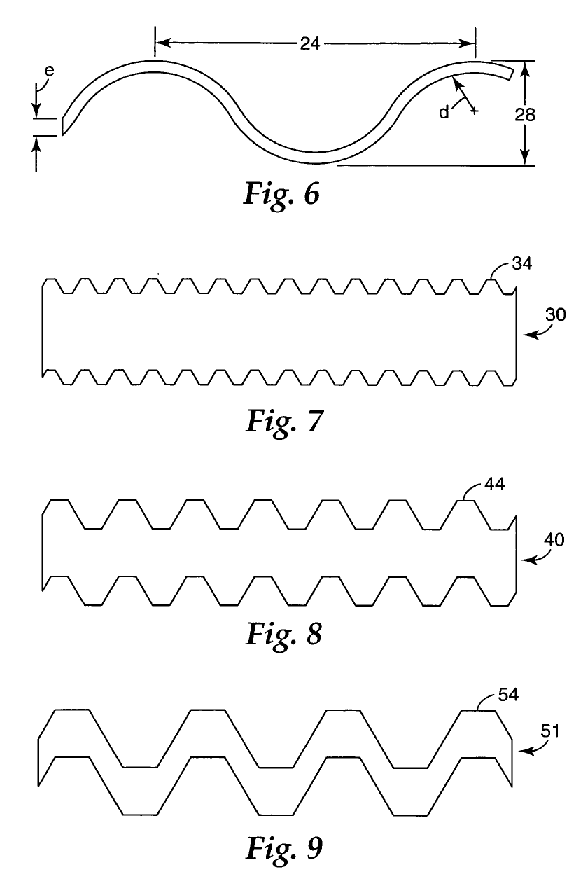

Variations of the FIG. 2 embodiment are shown in FIGS. 3-5 with variations in the height 23 of the features 22 and the distance between features 24. With the FIG. 6 embodiment, the width of the feature ‘e’ relative to the landing member width 28 is too small resulting in a landing member with low functional performance and low dimensional stability.

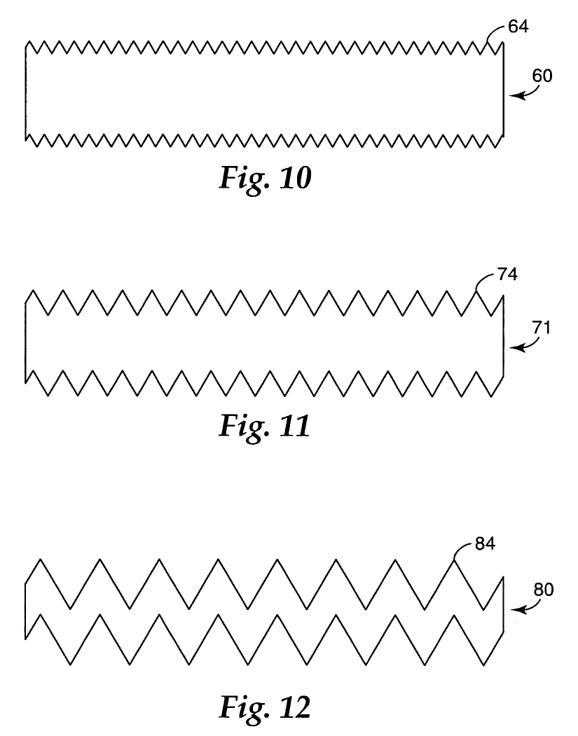

Alternative variations of the invention landing member 30, 40, 51, 60, 71, 80 using different shapes of features 34, 44, 54, 64, 74, 84 are shown in FIGS. 7-12. The softening effects and reference functions are met using these feature shapes. These shapes could be used alone or in any combination such as the FIG. 7 shape used with the FIG. 12 shape or any other combination. For example, visual referencing could be enhanced by using a combination of feature shapes in a defined order with a central feature with a defined set of features on each side of the central feature, which constantly change going from the central feature to an edge 29a, 29b of the landing member. The variation could be in shape, height, spacing or some combination thereof.

### Test Methods

135 Degree Peel Test

A 135 degree peel test was used to measure the amount of force that was required to peel a sample of fastening tape (KN-1520, available from 3M Company, St. Paul, Minn.) from a sample of film-based landing member tape consisting of release-coated biaxially oriented polypropylene test tape (KN-3829, available from 3M Company, St. Paul, Minn.). A 40 mm by 76 mm strip of the fastening tape was cut with the long dimension being in the transverse direction of the web. A 25 mm wide paper leader was attached to one end of the tape strip. A 40 mm by 127 mm rectangular sample (as a comparative) of the oriented test tape was cut and adhered adhesive side down, to a 51 mm by 127 mm stainless steel panel. A shaped-edge landing member was also cut and tested. The fastening tape strip with adhesive side down, was placed onto the release-coated surface of the oriented test tape. The long axis of the fastening tape was parallel to the long axis of the steel panel. A 2 kg rubber-covered mechanically operated roller conforming to PSTC Test Methods, Appendage B, was used to adhere the fastening tape strip to the release surface of the oriented test tape by passing the roller over the laminate assembly once forward and once backward. The laminate assembly was held firm against the surface of a 135 degree jig stand mounted into the lower jaw of an INSTRON Model 1122 tensile tester. The loose end of the paper leader attached to the fastening tape strip was placed in the upperjaw of the tensile tester. A crosshead speed of 30.5 cm per minute was used to record the peel force as the fastening tape strip was peeled from the release surface of the oriented test tape at a constant angle of 135 degrees. An average of the four highest force peaks was recorded in grams and was reported in grams/2.54 cm-width. 7 replicates were tested with the average of the 7 being reported in Table 3 below.

Dynamic Shear Strength

The shear strength of a mechanical fastener hook material (KN-2536, available from 3M Company, St. Paul, Minn.) to a shaped-edge loop landing member was measured using a INSTRON Model 1122 tensile tester. The hook material was cut into strips 40 mm by 13 mm, the long direction being in the machine direction of the web. Strapping tape (SCOTCH 898 available from 3M Company, St. Paul, Minn.) was used to provide a test leader for the hook strip by cutting a 95 mm by 40 mm piece of the strapping tape, laminating it to the hook strip and then folding over the strapping tape onto itself to cover the remaining exposed adhesive. Loop landing member test samples were prepared using a commercially available loop material (KN-3680, 3M Company, St. Paul, Minn.). Loop test samples were cut into various shapes using a 40 mm by 127 mm rectangle as a starting point. The long edges of the loop rectangle were cut by hand using a razor into various patterns as described in the examples below. The loop test sample was reinforced using SCOTCH 898 strapping tape adhered to the film side of the test sample. Any exposed adhesive was trimmed away by hand such that the reinforcing tape pattern matched the test sample pattern. The hook strip test sample was carefully centered onto the loop test sample and a 5 kg hand held steel roller (6 cm radius, 57 mm width) was then rolled over the back of the hook strip five times in each direction at a rate of 305 mm/min. The sample was then placed in the jaws of the tensile tester with the free end of the strapping tape leader gripped in the upper jaws and the free end of the loop test sample gripped in the lower jaws. A jaw separation of 51 mm was used. A crosshead speed of 305 mm/min was used to separate the hook strip from the loop test sample in a shear mode. The maximum peak force was recorded in grams. 7 replicates were tested and averaged.

180 Degree Peel Strength

The peel strength of a mechanical fastener hook material (KN-2536, available from 3M Company, St. Paul, Minn.) to a shaped loop landing member was measured using a INSTRON Model 1122 tensile tester. The hook material was cut into strips 40 mm by 13 mm, the long direction being in the machine direction of the web. Using double-coated tape, the hook strip was laminated to the center of a 216 mm by 40 mm paper (20 pound) leader strip. The long direction of the hook strip was aligned in the short direction of the leader. A reinforcing staple was provided approximately 10 mm from one end of the leader. The opposite end of the leader was then folded over and behind the hook strip such that the fold line coincided with one edge of the hook strip. Loop landing member test samples were prepared using a commercially available loop material (KN-3680, 3M Company, St. Paul, Minn.). Loop test samples were cut into various shapes using a 40 mm by 127 mm rectangle as a starting point. The long edges of the loop rectangle were cut by hand using a razor into various patterns as described in the examples below. The hook strip test sample with leader attached was carefully centered onto the loop test sample and a 2 kg rubber-covered hand held roller conforming to PSTC Test Methods, Appendage B, was then rolled over the back of the hook strip once in each direction by hand. A large metal clamp was then attached to the stapled end of the paper leader. While holding one end of the loop test sample, a 1 kilogram weight was attached to the clamp at the end of the paper leader for 10 seconds to further engage the hook strip into the loop test sample. The hook/loop test assembly was then immediately tested in the tensile tester with one end of the loop test sample being in the upper jaws and the unstapled end of the paper leader being in the lower jaws. A jaw separation of 51 mm was used. A crosshead speed of 305 mm/min was used to separate the hook strip from the loop test sample in a 180 degree peel mode. The maximum peak force was recorded in grams. 7 replicates were tested, averaged and reported in Table 1 below.

Compression Stiffness

A compression test was used to measure the stiffness of the shaped loop-based and film-based landing members. Loop test samples were prepared using a commercially available loop material (KN-3680, 3M Company, St. Paul, Minn.). Film-based landing members were prepared using a commercially available frontal film tape (KN-3829, available from 3M Company, St. Paul, Minn.). Test samples were cut into various shapes using a 25 mm by 76 mm rectangle as a starting point, the long direction being in the cross or transverse direction of the web. One of the long edges of the sample rectangle was cut by hand using a razor into various patterns as described in the examples below. One of the edges was left uncut (i.e. straight) to provide a more stable foundation for the test procedure. A cylinder was formed from this sample by bringing together the two ends, overlapping them by approximately 4 mm, and then stapling the two ends together with 2 staples each approximately 3 mm from the top and bottom edges of the cylinder. The cylindrical sample was then placed on a platen mounted to the lower jaw of an INSTRON Model 5500R constant rate of extension tensile machine. The upper jaw of the tensile machine was equipped with a flat compression plate. This plate was lowered at a rate of 10 mm/min. The load to compress the sample was continuously recorded. The peak recorded load, which represents the force required to buckle the sides of the sample cylinder, is reported in kg/mm in Tables 2 and 4 below as Compression Stiffness. 5 replicates were tested and averaged.

Static Shear Adhesion

The shear adhesion of a piece of fastening tape laminated to a release-coated biaxially oriented polypropylene film test tape (KN-3829, available from 3M Company, St. Paul, Minn.) was measured using the following procedure. A 40 mm by 25 mm piece of fastening tape (KN-1520, available from 3M Company, St. Paul, Minn.), the long direction being in the cross or transverse direction of the tape, was laminated to a 40 mm by 127 mm rectangular piece of the biaxially oriented polypropylene test tape, the long direction being in the machine direction of the tape. A shaped-edge film-based landing member tape was also tested by cutting a sinusoidal-type pattern into the edges of the tape as shown in FIG. 3. A reinforcing tape (KN-1759, available from 3M Company, St. Paul, Minn.) was laminated to the test tape to enhance the overall stiffness for testing purposes. A 2 kilogram rubber covered hand held roller conforming to PSTC Test Methods, Appendage B, was passed over the laminate once in each direction at 30.5 cm per minute to firmly adhere the fastening tape to the polypropylene test tape. The two-tape laminate was clamped at one end and hung vertically in a 40° C. oven for 15 minutes after which a 1 kilogram weight was attached to the loose bottom end of the laminate, generating a shear load at a 180° angle. The time that it took in minutes for the weight to drop at 40° C. was recorded as a measure of the shear adhesion. Reported values are averages of 7 tests. The test was discontinued after 2 weeks (20,000 minutes) if shear failure had not occurred.

### EXAMPLE 1

A shaped-edge loop-based landing member was prepared using a commercially available loop material (KN-3680, 3M Company, St. Paul, Minn.). A 40 mm by 127 mm rectangular patch was cut from a roll of material after which the long edges of the rectangular loop strip were cut into a sinusoidal-type edge by hand using a razor into the pattern shown in FIG. 2 having dimensions 28=40 mm, 23=3.2 mm, 24=11 mm, and ‘d’=3.2 mm radius. The area of the shaped landing member was 46.8 cm² as compared to the starting rectangular area of 50.8 cm², resulting in a 7.9% area reduction. The peel, shear and compression stiffness properties of the shaped patch are shown in Tables 1 and 2 below along with comparatives of a straight-edged rectangular landing member.

### EXAMPLE 2

A shaped loop landing member was prepared as in Example 1 except a more exaggerated edge pattern was used as shown in FIG. 3 and having dimensions 28=40 mm, 23=6.4 mm, 24=22 mm, and ‘d’=6.4 mm radius. The area of the shaped landing member was 42.7 cm² as compared to the starting rectangular area of 50.8 cm², resulting in a 15.9% area reduction. The peel, shear and compression stiffness properties of the shaped landing member are shown in Tables 1 and 2 below along with comparatives of a straight-edged rectangular landing member.

### EXAMPLE 3

A shaped loop landing member was prepared as in Example 1 except a more exaggerated edge pattern was used as shown in FIG. 4 and having dimensions 28=40 mm, 23=12.7 mm, 24=44 mm, and ‘d’=12.7 mm radius. The area of the shaped landing member was 34.7 cm² as compared to the starting rectangular area of 50.8 cm², resulting in a 31.7% area reduction. The peel, shear and compression stiffness properties of the shaped landing member are shown in Tables 1 and 2 below along with comparatives of a straight-edged rectangular landing member.

### EXAMPLE 4

A shaped loop landing member was prepared as in Example 1 except a more exaggerated edge pattern was used as shown in FIG. 5 and having dimensions 28=40 mm, 23=18 mm, 24=62 mm, and ‘d’=18 mm radius. The area of the shaped landing member was 27.9 cm² as compared to the starting rectangular area of 50.8 cm², resulting in a 45% area reduction. The peel, shear and compression stiffness properties of the shaped landing member are shown in Tables 1 and 2 below along with comparatives of a straight-edged rectangular landing member.

### COMPARATIVE EXAMPLE C1

A shaped loop landing member was prepared as in Example 1 except a more exaggerated edge pattern was used as shown in FIG. 6 and having dimensions 28=40 mm, ‘e’=5 mm, 24=121 mm, and ‘d’=35 mm radius. The area of the shaped landing member was 6.4 cm² as compared to the starting rectangular area of 50.8 cm², resulting in a 87.4% area reduction. The peel, shear and compression stiffness properties of the shaped landing member are shown in Tables 1 and 2 below along with comparatives of a straight-edged rectangular landing member. To provide adequate rigidity and meaningful test results for the peel and shear tests, the shaped landing member was laminated to the smooth film side of a 50 mm by 70 mm piece of KN-3680 loop material (3M Company, St. Paul, Minn.) prior to testing. The 180 degree peel test was done using a 200 gram weight to further engage the hook strip into the loop sample instead of a 1 kilogram weight due to the significantly lower tensile strength of the shaped loop landing member. As seen in Table 1, Comparative Example C1 does not provide adequate peel and shear strength due to the significantly lower hook/loop contact area.

### EXAMPLE 5

To demonstrate a shaped landing member using a film-based landing member, a shaped landing member was prepared as in Example 2 except a biaxially oriented polypropylene tape (KN-3829, available from 3M Company, St. Paul, Minn.) was used in place of the loop material. The long edges of the tape were cut to the pattern as shown in FIG. 3 and having dimensions 28=40 mm, 23=6.4 mm, 24=22 mm, and ‘d’=6.4 mm radius. The area of the shaped landing member was 42.7 cm² as compared to the starting rectangular area of 50.8 cm², resulting in a 15.9% area reduction. The peel, shear and compression stiffness properties of the shaped landing member are shown in Tables 3 and 4 below along with comparatives of a straight-edged rectangular landing member. The displacement at peak force was not recorded.

TABLE 1

| | Hook/loop | | |
|---|---|---|---|
| Landing | Contact area | 180 degree Peel | Dynamic Shear |
| Member | (mm²) | Strength (grams) | Strength (grams) |
| Rectangular | 520 | 913 | 10160 |
| comparative | | | |
| Example 1 | 479 | 922 | 9324 |
| Example 2 | 437 | 863 | 8850 |
| Example 3 | 355 | 674 | 6100 |
| Example 4 | 286 | 565 | 5047 |
| Comparative | 65 | 70 | 994 |
| Example C1 | | | |

TABLE 2

| | Compression Stiffness | |
|---|---|---|
| | | Displacement at |
| Landing Member | Peak Force (grams) | Peak Force (mm) |
| Rectangular | 602 | 0.6 |
| comparative | | |
| Example 1 | 482 | 5.0 |
| Example 2 | 341 | 8.7 |
| Example 3 | 131 | 9.4 |

TABLE 3

| | Hook/loop | 135 degree | Static Shear |
|---|---|---|---|
| Landing | Contact area | Peel Strength | Adhesion |
| Member | (mm²) | (grams) | (minutes) |
| Rectangular | 520 | 503 | >20,000 |
| comparative | | | |
| Example 5 | 437 | 507 | >20,000 |

TABLE 4

| | Compression Stiffness | |
|---|---|---|
| | | Displacement at |
| Landing Member | Peak Force (grams) | Peak Force (mm) |
| Rectangular | 132 | — |
| comparative | | |
| Example 5 | 43 | — |

Although particular embodiments and/or individual features of the present invention have been illustrated and described, it would be obvious to those skilled in the art that various other changes and modifications can be made without departing from the spirit and scope of the invention. Further, it should be apparent that all combinations of such embodiments and features are possible and can result in preferred executions of the invention. Therefore, the appended claims are intended to cover all such changes and modifications that are within the scope of this invention.

We claim:

1. A disposable absorbent article comprising:
(a) a chassis having a longitudinal centerline and an outwardly-facing surface and an inwardly-facing surface, the chassis adapted to extend from a back waist area of a wearer to a front waist area of a wearer with the inwardly-facing surface overlying the crotch area of the wearer, the chassis including a first back waist region, a second front waist region and a crotch region; and
(b) a fastening system comprising at least one first fastening element and a landing member engagable with the at least one first fastening element on opposing waist regions wherein the landing member has a top edge and a bottom edge wherein at least the top edge is nonlinear and provided with a multitude of features.

2. The disposable absorbent article of claim 1 wherein the landing member is on the second front waist region.

3. The disposable absorbent article of claim 2 wherein the landing member is centered on the longitudinal centerline.

4. The disposable absorbent article of claim 3 wherein the landing member features are generally symmetrically arranged on either side of the centerline.

5. The disposable absorbent article of claim 2 wherein the features are a series of peaks and valleys.

6. The disposable absorbent article of claim 2 wherein the landing member has a continuous backing.

7. The disposable absorbent article of claim 2 wherein the landing member has a tensile strength of at least 0.5 kg/2.54 cm.

8. The disposable absorbent article of claim 6 wherein the landing member has a tensile strength of at least 1.0 kg/2.54 cm.

9. The disposable absorbent article of claim 6 wherein the features are spaced at a distance of at least 2 mm.

10. The disposable absorbent article of claim 2 wherein the features are spaced at a distance of from 5 to 100 mm.

11. The disposable absorbent article of claim 9 wherein the features have a height of at least 2 mm.

12. The disposable absorbent article of claim 10 wherein the features have a height of at least 3 mm.

13. The disposable absorbent article of claim 11 wherein the ratio of the height of the features to the width between the peaks is from 0.02 to 10.

14. The disposable absorbent article of claim 12 wherein the ratio of the height of the features to the width between the peaks if from 0.03 to 5.0.

15. The disposable absorbent article of claim 13 wherein the features have radii of curvature of greater than 2 mm.

16. The disposable absorbent article of claim 14 wherein the features have radii of curvature of greater than 3 mm.

17. The disposable absorbent article of claim 2 wherein the landing member bottom edge has features.

18. The disposable absorbent article of claim 2 wherein the width of the landing member is generally greater than or equal to the width of the fastening element.

19. The disposable absorbent article of claim 2 wherein the landing member has a Gurley Stiffness of from 6 to 500.

20. The disposable absorbent article of claim 6 wherein the landing member has a Gurley Stiffness of from 10 to 500.

21. The disposable absorbent article of claim 20 wherein the landing member has a compression stiffness of less than 700 grams.

22. The disposable absorbent article of claim 2 wherein the height of the features is from 5 to 50 percent of the maximum landing member width.

23. The disposable absorbent article of claim 6 wherein the height of the features is from 10 to 40 percent of the maximum landing member width.

24. The disposable absorbent article of claim 22 wherein the height of the features is greater than 2 mm.

25. The disposable absorbent article of claim 23 wherein the height of the features is greater than 3 mm.

26. The disposable absorbent article of claim 24 wherein the features reduce the compression resistance of at least a portion of the landing member top edge by at least 15 percent.

27. The disposable absorbent article of claim 24 wherein the features reduce the compression resistance of at least a portion of the landing member top edge by at least 30 percent.

28. The disposable absorbent article of claim 24 wherein the features reduce the compression resistance of at least a portion of the landing member top edge by at least 50 percent.

29. The disposable absorbent article of claim 2 wherein the features are irregular in size and shape.

30. The disposable absorbent article of claim 4 wherein the features are irregular.

31. The disposable absorbent article claim 30 wherein the features size and shape are symmetrical on either side of the centerline.
